(19) United States
(12) Patent Application Publication
(10) Pub. No.: US 2024/0197564 A1
(43) Pub. Date: Jun. 20, 2024

(54) METHOD AND APPLICATION PERSONALIZING ACUPRESSURE FOR IMPROVED WELLNESS

(71) Applicant: 5 Point App, Inc., New York, NY (US)

(72) Inventors: Ryan SMITH, Greenwood Lake, NY (US); Arthur MANDELL, New York, NY (US); Susan L. BECK, Forth Mill, SC (US); Gary RIGER, Mineola, NY (US); Derek MERDINYAN, Issaquah, WA (US); Michael FAGBAMIGBE, Holtsville, NY (US)

(73) Assignee: 5 Point App, Inc., New York, NY (US)

(21) Appl. No.: 18/545,769

(22) Filed: Dec. 19, 2023

Related U.S. Application Data
(60) Provisional application No. 63/476,145, filed on Dec. 19, 2022.

(51) Int. Cl. A61H 39/04; G16H 10/60; G16H 15/00; G16H 20/40; G16H 20/90; G16H 50/70

(52) U.S. Cl. CPC A61H 39/04; G16H 10/60; G16H 15/00; G16H 20/40; G16H 20/90; G16H 50/70

(57) ABSTRACT

A method and application for enhancing patient wellness assists patients for self-application of acupressure by collecting personalized data from the patent relating to such matters as individual sleep and activity, instructing the patient on procedures for self-application of acupressure, and monitoring the patient's symptom response such as energy levels, activity and sleep. In this way, the acupressure treatment and provide can be assessed to provide treatment data for feedback to the patient. Such treatment data from multiply patents can be collected to enhance research and further improve acupressure treatment modalities.

### CROSS-REFERENCE TO RELATED APPLICATION

This application claims the benefit of priority to U.S. Provisional Ser. No. 63/476,145 filed Dec. 19, 2022. The drawings and descriptions of the provisional application are incorporated herein by this reference.

### FIELD OF THE INVENTION

The present invention relates to personalizing acupressure for enhanced wellness by not only instructing the user in self-administration of acupressure, but also enabling personalized information from the user to be received and compared with earlier data to provide updated information to the user and, in conjunction with user feedback, provide information to enhance self-application of acupressure. Not only can the wellness of the user be improved, but data can be compiled from many users to assist further research into improvements for acupressure treatment.

### BACKGROUND OF THE INVENTION

Acupressure is the modality of applying pressure by either a person's fingers or a device to specified acupressure points on the body known as acupoints. Inasmuch as acupressure has been known to reduce chronic pain and fatigue, as well as improve sleep for greater energy and wellness, acupressure can be of assistance in treating adverse symptoms of cancer and its treatment, or other serious illness. Known applications for self-administration of acupressure often display instructional diagrams and videos, but do not collect and analyze data to provide guidance to further enhance treatment of the patient, nor collect and analyze such data to advance treatment modalities and options for use of acupressure.

It is, therefore, an object of the present invention to provide enhanced instruction to the patient of use of acupressure and monitor the results and collect data to enhance further use by the patent of acupressure as well as support research to advance treatment modalities.

### SUMMARY OF THE INVENTION

These and other objects, features and advantages of the present invention are provided by a method and application for enhancing patient wellness by assisting patients for self-application of acupressure by collecting personalized data from the patent relating to such matters as individual sleep and activity, instructing the patient on procedures for self-application of acupressure, and monitoring the patient's symptom response such as energy levels, activity and sleep to assess the acupressure treatment and provide valuable treatment data for feedback to the patient. Such treatment data from multiply patents can be collected to enhance research and further improve acupressure treatment modalities.

Preferably, the user may wear a health monitor able to sense data directly from the user, and data input by the user, are transmitted to a central database to identify acupoints and modalities useful to the patient's treatment, and the degree, placement and manner of pressure preferred to be applied in further treatments.

These and other objects, advantages and features of the present invention will become apparent from the detailed descriptions made below with reference to the accompanying diagrams.

### BRIEF DESCRIPTION OF THE DRAWINGS

FIG. 1 is a flowchart of the basic operating principles of the present invention;

FIG. 2 is a flowchart of the launch sequence of the present invention;

FIG. 3 is a flowchart of the welcome sequence of the present invention;

FIG. 4 is a flowchart of the user authentication sequence of the present invention;

FIG. 5 is a flowchart of the post-launch sequence of the present invention;

FIG. 6 is a flowchart of the post-registration sequence of the present invention;

FIG. 7 is a flowchart of the profile sequence of the preset invention;

FIG. 8 is a flowchart of the profile-creation sequence of the present invention;

FIG. 9 is a flowchart of the heath survey sequence of the present invention;

FIG. 10 is a flowchart of the entry of National Institute of Health's Patient-Reported Outcomes Measurement (PROMIS) information for the present invention;

FIG. 11 is a flowchart of the dashboard sequence for the present invention that provides visuals for the user;

FIG. 12 is a flowchart of the ritual for the patient applying accupressure of the present invention;

FIG. 13 is a flowchart of the display to the user of the statistics from use of the present invention;

FIG. 14 is a flowchart of the procedures for the user participating in a community of users of the preset invention;

FIG. 15 is a flowchart of the account procedures for the user of the present invention ritual for the patient applying acupressure of the preset invention;

FIG. 16 is a flowchart of the instructions provided to the user to perform a stimulating or relaxing acupressure ritual;

FIG. 17 is a flowchart of the use of the present invention with a community.

### DETAILED DESCRIPTION OF PREFERRED EMBODIMENT

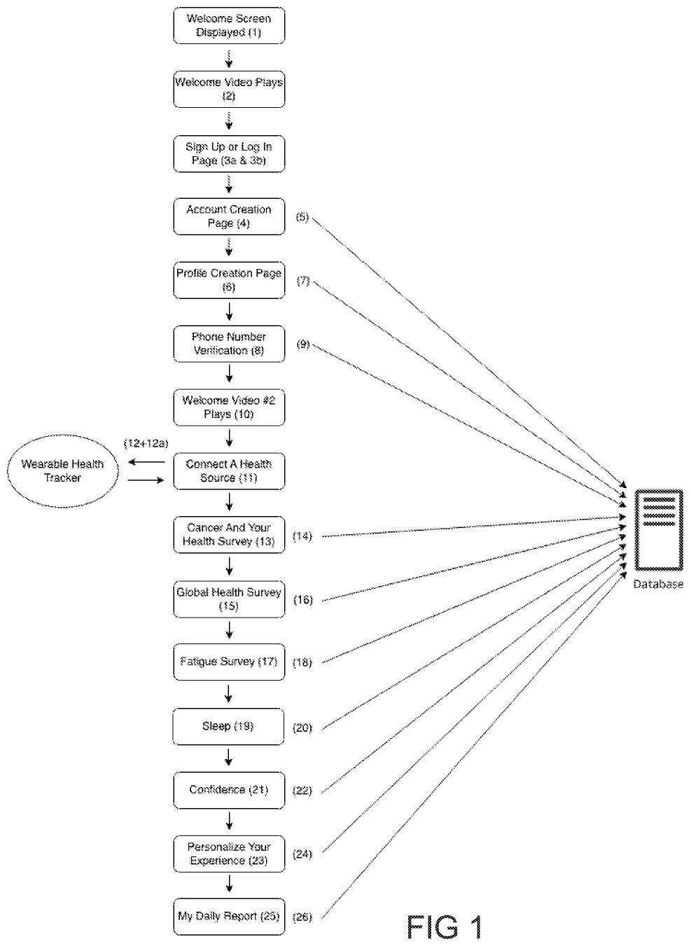

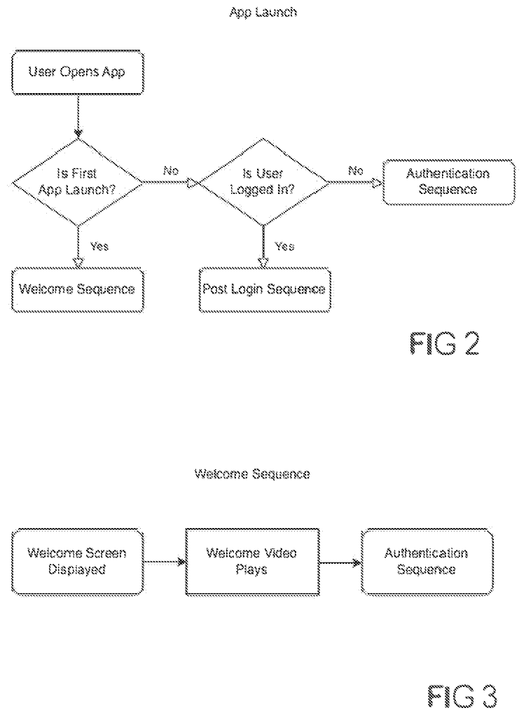

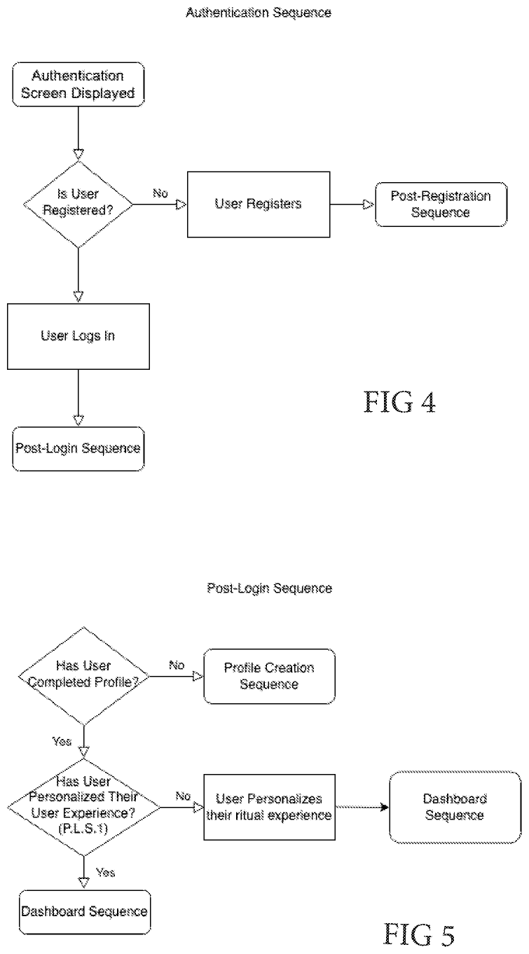

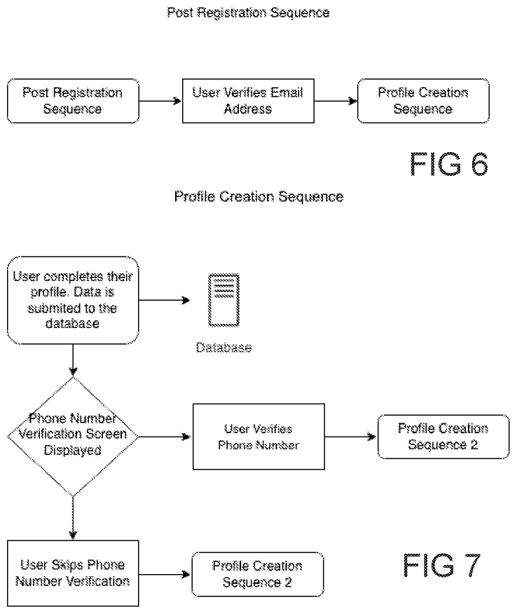

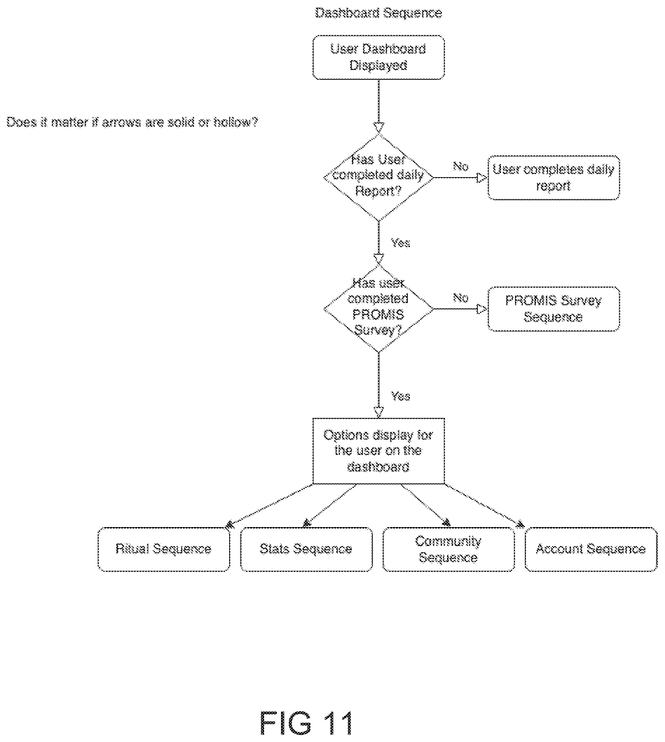

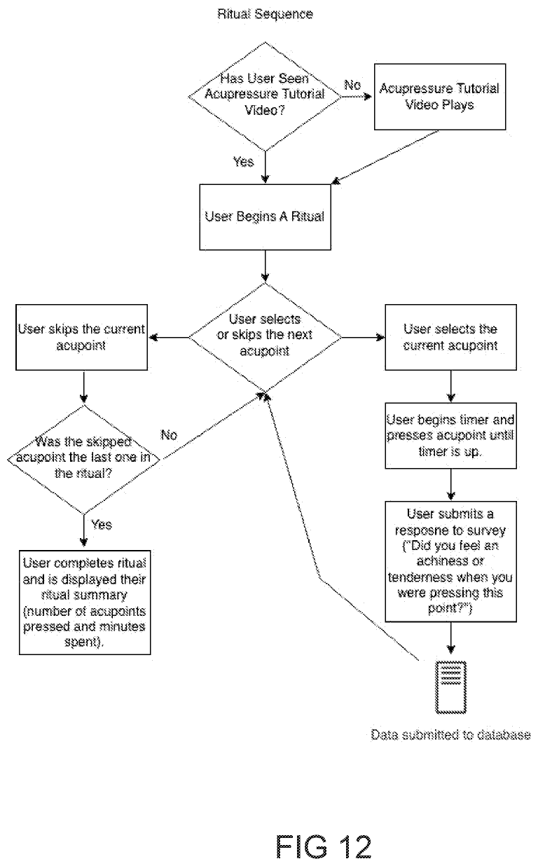

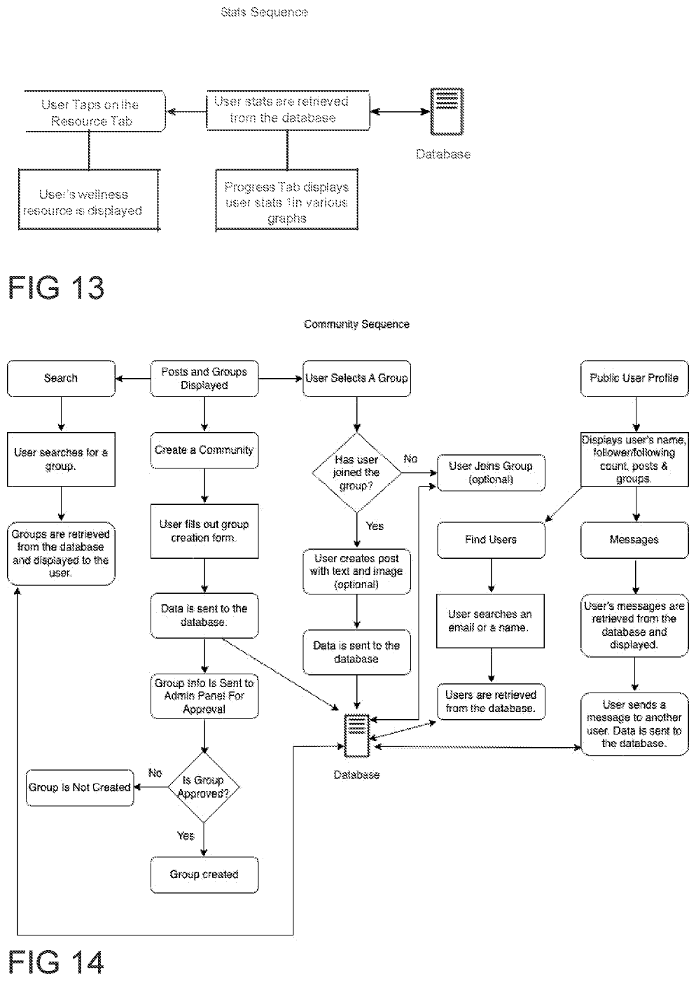

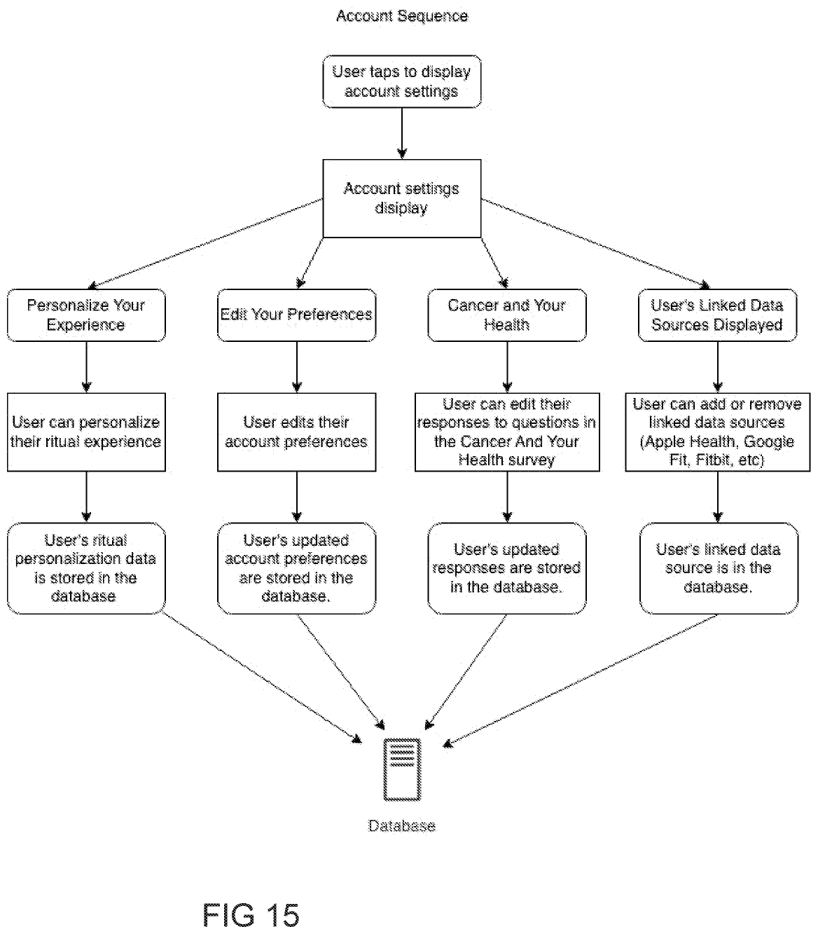

The flow diagrams of FIGS. 1 through 17 illustrate the operation of the present invention, and would be well understood by one skilled in the field of developing software for applications. The present invention is intended to be used with a health monitor such as a fitness tracker worn by the user, and the application monitors daily energy levels and symptom response to acupressure treatment. Alternatively, the user could enter data directly without the use of a health monitor. A remote database is envisioned to accumulate the data for each user, and to control the operation. The present invention can therefore be implemented as a software application or “app.”

The present invention also provides instructions, preferably by presenting videos, to the user explaining acupressure to better manage pain, fatigue and improve sleep by use of the present invention, and how to self-administer acupressure. The present invention also can connect with a health data source such as Apple Health, Google Fit, Fit Bit and other sources of collecting health data.

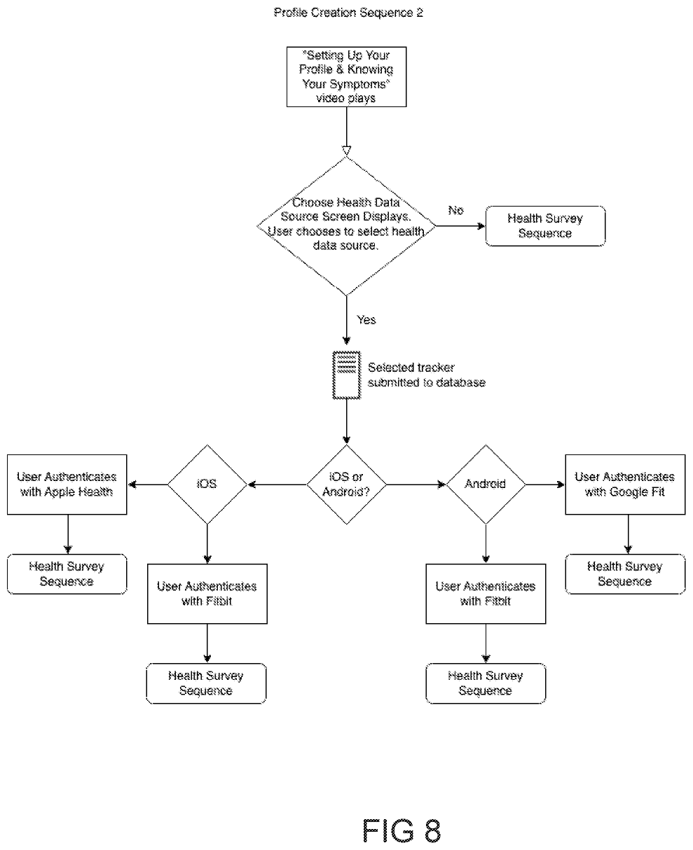

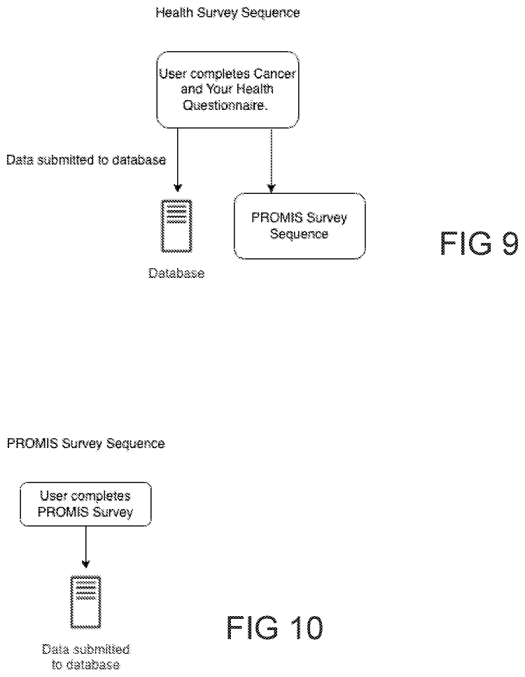

When setting up a profile, the user will enter the Health Survey Sequence shown in FIG. 9 to provide information about diagnosis and treatment of an illness such as cancer, and the use of pharmacologic and nonpharmacologic approaches to improve fatigue and sleep deficiency, as shown in FIG. 8. The questions asked about health and treatment, and fatigue are based upon the National Institute of Health's Patient-Reported Outcomes Measurement Information System (PROMIS) criteria. The present invention also presents in an easy-to-understand manner graphic representations of the progress the user will be experiencing from use of the present invention.

And the data generated by each user will be collected by the database and organized to be used for research purposes to further enhance the use of acupressure to treat symptoms of major diseases or the side effects of their treatment. And groups can be formed of users having commonalities to enhance the positive effects of use of the present invention as explained in FIG. 14.

In this way, the user can use the present invention to track and monitor symptoms resulting from cancer or the treatment of cancer, such as lower levels of energy, sleep loss, nausea and the like. The present invention, however, can be used to track and compile data for any number of symptoms that can be positively affected by acupressure. Use of the present invention for treatment of cancer-related symptoms is illustrative.

An important part of the present invention is the instruction provided to the user. The user is surveyed on confidence of use of acupressure and the ability to use the application for acupressure rituals each day or another period of use. Personal data collected includes individual sleep and activity data that can be input by the user directly, or synced from a health monitor such as a fitness tracker worn by the user. The application monitors daily energy levels and symptom response to acupressure treatment. In this way, the user can use acupressure for such purposes as to better manage fatigue and improve sleep by monitoring self-reports, the user's activity and data from the fitness tracker and information received from other users as well as known treatment data, all supplied to the common database.

To track progress, a baseline energy level is established, which should increase as acupressure rituals are performed. Patients tend to benefit from being active and confident managers of their health. Cancer and cancer treatment, as well as other serious illnesses, can cause a number of issues: fatigue, sleeplessness, anxiety, depression and pain. The present invention helps such patients better understand their symptoms and the relationship of such symptoms to overall energy and wellbeing.

The present invention establishes baseline energy levels with self-reporting symptoms through survey questions and in addition, the present invention enables patients to track the percentage of each acupressure session completed, as well as tracks gains and the acupressure use to demonstrate to each patient how use of the present invention translates to symptom improvement and overall wellbeing. The self reports from the survey and the data from the patient's fitness tracker collecting data on the hours of sleep each night and daytime activity as measured by step count and brisk exercise, and the like, as well understood in the fitness tracker art.

The patient is presented with a choice to connect a health monitor, such as a fitness tracker, and allow access to existing programs such as Apple Health, Google fit, connect to a Fitbit, or skip connecting; and the patient's selected tracker data is sent to the database. The data is updated at least daily per connected health tracker, and maintained in the database. The patient is provided with a survey for information concerning the patient's overall medical history, and specifically about any cancer, or other serious illness, diagnosis and treatment and use of other pharmacologic and nonpharmacologic approaches to improve fatigue and sleep deficiency. The responses are stored in the database.

The patient is also surveyed about their fatigue using National Institute of Health's Patient-Reported Outcomes Measurement (PROMIS) to validate patent reported outcomes of individuals living with chronic conditions.

Surveys are also taken of sleep habits and experiences, and about the patient's confidence in an ability to perform acupressure accurately upon themselves. Additionally, personal information is requested whether the user wants to perform the relaxing acupressure ritual, the stimulating acupressure or both acupressure rituals each day. User also indicates whether or not they are going to use essential oils while performing acupressure on themself. Based on these choices, instructional and informational videos are provided to the user.

These videos discuss acupressure and contraindications from using the acupressure techniques explained to the user. The videos note acupressure is considered safe with possible side effects, such as bruising. The patent is advised acupressure should never be applied directly to a tumor and to avoid pressing on a point that may be near a wound, scar or sensitive area. The patent is advised traditional East Asian medicine knowledge provides the framework for the two acupressure rituals that can be used by the present invention in order to help with fatigue and sleep and improve adverse symptoms.

The first traditional East Asian medicine technique is stimulating acupressure that can strengthen the body's energy production system. The second traditional East Asian medicine technique is relaxing acupressure, which bolsters energy by activating the body's restorative and relaxation systems. The present invention through accessed video demonstrations and graphic instructions teaches the user how to implement both stimulating and relaxing acupressure, and both techniques are recommended as simulating acupressure and relaxing acupressure can significantly reduce symptoms of severe illness by modulating brain neural pathways and improving sleep. The relaxing ritual has been found to notably improve sleep quality. Conversely, the stimulating ritual acts on regions that may increase alertness.

The present invention also provides a video for the proper use of essential oils, notable aromatherapy, as use of pleasant-smelling essential oils can improve wellbeing. The present invention instructs by selected videos on use of essential oils, as well as essential oil safety and contraindications; and application methods such as inhalation and topical.

The present invention also obtains daily surveys to obtain user information concerning health and feelings of wellbeing, either by the fitness tracker or requesting user manual input. Such questions as rate the quality of the user's previous day, rate the previous night's sleep and fatigue. The results are tabulated in the database and presented as a polar graph to the user with supportive messages encouraging users to stay with consecutive days of acupressure.

The present invention includes a “Dashboard” easily visible to the user that contains supportive messages encouraging users to stay on their “streak” of consecutive days of acupressure. It also contains a Polar Graph where all measures are contained as pieces of an “Energy Pie.” Each “pie slice” is a different measure, or “energy contribution,” so users can see their energy contributions at a glance and determine where they may need more of a certain energy contribution.

Another data visual can be presented whereby each energy contribution is represented by a bar graph and an up or down arrow depending on if that energy contribution has increased or decreased in the last day. Users can toggle to a “Stats Board” to see all their energy contributions in line graph form and better understand how each energy contribution changes over time. The pie graph at the top of the Dashboard allows the user to see all of the energy contributions at a glance. Each part of the graph grows larger as energy contributions improve. The favorable energy contributions are less fatigue—shown in red, better sleep—shown in orange, improved physical and mental health—shown in green, increased activity—shown in blue, and completion of the acupressure rituals—shown with purple.

These energy contributions, represented by the pie pieces, are also represented as line and bar graphs below the polar graph. The user can thus have a visual representation how each energy measure fluctuates over time. In a line graph, time, in days or weeks, can be shown on the horizontal line. Energy measure is shown on the vertical line. The user can toggle to see data for previous days or weeks. For example, daily fatigue shows a daily snapshot of fatigue as it changes day by day and weekly fatigue shows how fatigue from the weekly PROMIS measures changes week by week. In a bar graph, the user can see how energy grows as the user completes a greater percentage of the guided acupressure sessions. With each session performed, the percent completion bar will grow to show the percentage of relaxing and stimulating rituals have completed since period.

The polar graph displays graphically with each segment being a different measure, or “energy contribution,” so users can see their energy contributions at a glance and determine where they may need more of a certain energy contribution.

The graphic presentation provides data visually whereby each energy contribution can be represented by a bar graph and an up or down arrow depending on if that energy contribution has increased or decreased in the last day, or last period for data input. Users can toggle to a “Stats Board” to see all their energy contributions in line graph form and better understand how each energy contribution changes over time. The energy contributions displayed indicate less fatigue, better sleep, improved physical and mental health, increased activity, and completion of the acupressure rituals all calculated by accepted algorithms known in the research field from assessed data supplied by survey answers and information from the fitness tracker or data input by the users. The user can easily see how each energy measure fluctuates over time. The user can toggle to see data for previous days or weeks. The user can see how energy grows as a greater percentage of the acupressure sessions are completed.

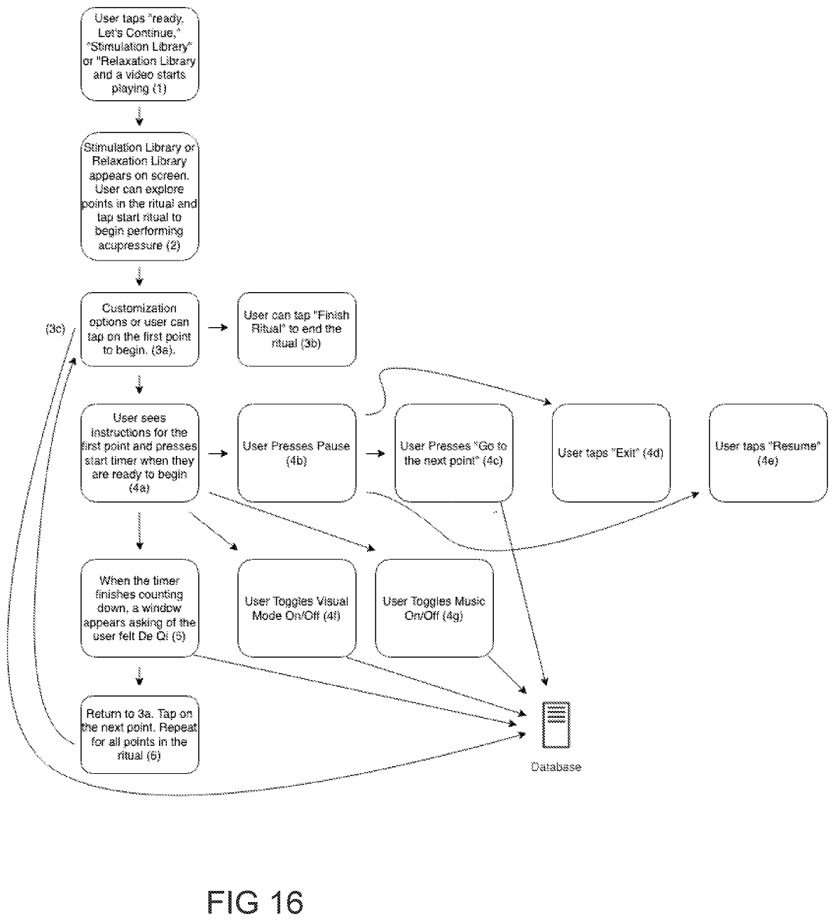

The present invention instructs the user to perform a stimulating or relaxing ritual, as outlined in FIG. 16.

The video accessed by the user provides instructions, describing and illustrating the acupoints using the images on the video provided for each acupressure ritual to be performed, including instructions for use of the appropriate essential oils if desired. The user is instructed to use the pad of a finger to apply firm, continuous pressure in a clockwise circular motion to each point identified to the user for three minutes or instructed to apply the eraser at the end of a pencil, or appropriate acupressure tool, on the acupoint instead if the fingers become tired or cramped.

The video explains the method of applying pressure to each acupoint, including the description of “de qi” describing a feeling of numbness, warmth, tingling, aching provided buzzing. As the user finishes each acupoint, the present invention will ask whether “qi” was felt; a positive response directing the user to the next acupoint.

The present invention presents to the user a “Stimulation Library” or the “Relaxation Library” on screen. User can tap on any of the illustrated acupoints to learn about how to find and press on the acupoints for that particular ritual. When the user taps, “Start Ritual,” the user is taken to a layout page for the ritual. The present invention demonstrates to a user how to perform self-acupressure. Alternatively, a “caregiver mode” is provided where the instructions (video, image, and text) teach care givers to perform acupressure on others.

If the user presses the ‘Pause Ritual’ button, a window displays options to keep the timer paused, they can skip to the next acupoint or they can exit the ritual. This allows the user to take a break in the middle of the acupressure session and resume it when they are ready.

If the user presses the ‘Go to next point’ button, they receive a short questionnaire asking “Did you feel achiness or tenderness while you were pressing this point?” The response, and the skipped status of the point, and the time the user spent pressing are sent to the databank. According to Traditional East Asian medicine (TEAM), de qi experienced by patients is often described as suan (aching or soreness), ma (numbness or tingling), zhang (fullness, distention, or pressure), and zhong (heaviness) and is felt by the acupuncturests (needle grasping) as tense, tight, and full. The input on the felt de qi is directed to the databank.

The de qi factor is considered an important variable in the studies of the mechanism and efficacy of acupressure treatment. Research is ongoing to understand de qi and its effect when analyzing treatment modalities. The present invention by including a couple of questionnaires to qualify and quantify de qi sensations, reviewing and incorporating neuroimaging studies of de qi and acupuncture and acupressure in the data bank, physiological mechanisms of de qi, and the relation between de qi and clinical efficacy, the present invention captures this information for each acupoint a user stimulates, preserves the data for use in further analysis and comparison of results, both with the user and the community of users.

The present invention includes a “Visual Mode”. Users can select the Visual Mode to display a repeating image along with a countdown of the timer for the viewer to watch while they are actively pressing the acupoint. The present invention stores a record in the database to keep track of when users activate Visual Mode during their acupressure sessions. Tracking visual mode allows to determine if this variable enhances users' experience of the present invention or improves their symptoms when used in addition to acupressure.

The present invention includes a “Music Mode” where the user can select to play or pause relaxing background music. The present invention stores information in the database of use of the Music Mode to keep track of when users apply music when performing acupressure on themselves and determines if music enhances their health outcomes.

At the end of the 3 minutes of applying pressure to an aupoint, a window appears asking the user if they noticed achiness or tenderness associated with the sensation of Qi. When the user submits a response, the answer is sent to the database along with a log that indicates whether the user pressed the acupoint for 3 full minutes. The actions taken, whether completed or not, are stored in the database for use in calculating and displaying energy levels.

The data recorded and analysis can be reviewed by the user of the present invention. The first time a user selects such information, labeled as “Stats” a video plays and explains how to view and understand the health stats and data presented. The information presented to the user in graphic form will illustrate how the user's health status has changed over time. The data presented illustrates the day and week results are being displayed and tracks the application of acupoint engagement. Dates can be presented to compare use from week to week or compare stats with others using the present invention.

The first, each day of use of the present invention, the user will be asked a few questions about sleep and energy levels. Secondly, once a week, the present invention will display the Weekly PROMIS surveys as accessed from the database to compare and measure the health and symptoms of the user based on standardized measures. Thirdly, data synced from the fitness tracker also provides important information about the user's activity and sleep patterns.

Graphic displays such as a pie graph will be presented to illustrate all of the energy contributions at a glance, and the graph is dynamic in that portions of the graph become larger as energy contributions improve. The energy contributions illustrated include less fatigue, better sleep, improved physical and mental health, increased activity, and completion of the acupressure rituals, all represented in different colors for easy review and understanding by the user.

These energy contributions, are also represented in a graphic display as line and bar graphs below the polar graph. The user is able, therefore, to easily understand how each energy measure fluctuates over time, and displays can be generated from the collected data to view the collected energy contributions for previous days or weeks. For example, daily fatigue levels can be displayed as a daily snapshot and illustrate changes day by day and weekly fatigue shows how fatigue varied from the weekly PROMIS measures changes week by week. A bar graph will display how energy contributions increase as the user completes a greater percentage of the acupressure sessions.

With each session accomplished, the percent completion bar on the graph will show the progress. For example, a bar graph can be created from the collected data to illustrate the percentage of relaxing and stimulating rituals completed, and the times they were completed.

Weekly Surveys are calculated by point values to the different answers of each of the PROMIS questions by PROMIS. The questions are asked and graded weekly. When a user views the graphs, the user's fatigue, sleep, health, and acupressure progress data is retrieved from the database and formatted to be displayed in the graph. Depending on which health data source the user has connected, additional sleep data (number of hours asleep, number of hours in bed, and number of times woken up) and activity data (step count & exercise time) is retrieved from the health monitor and data is retrieved from the relevant data source to be displayed to the user where accessed.

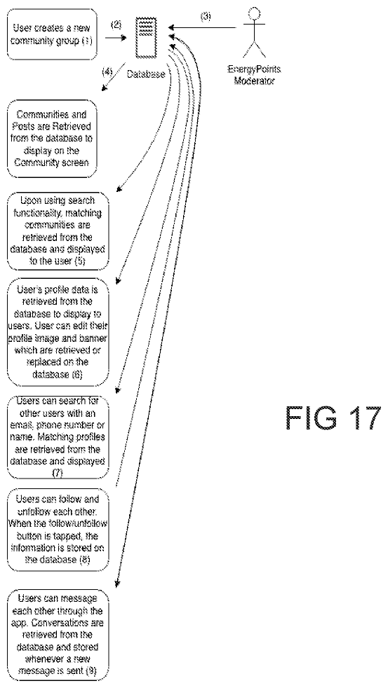

The present invention also includes a social or community component that allows users to create communities in which other users can join, create posts with text and/or an image and comment on posts. This feature enables virtual support groups, patient advocacy groups, and information sharing, educational groups that can benefit patient outcomes. This community feature operates as shown in FIG. 17.

The community group is reviewed and approved by a moderator. Users have a public profile which displays their name, description, profile image, banner image, follower count, following count, groups they are in, and posts they have created; all of which is retrieved from the database. Users can edit the profile image and banner image which gets sent to and retrieved from the database upon edit. Users can search for other users with an email or a name. Upon search, matching user profiles are retrieved from the database and displayed.

Users can follow (and unfollow, if already following) one another. Users can message one another through direct messaging. Conversations are retrieved from the database and stored whenever a new message is sent in a conversation.

A user of the present invention is able to personalize use of the program. Users can modify their daily ritual sessions and switch between 1 ritual per day (Stimulation or Relaxation) or 2 rituals per day (Stimulation and Relaxation).

The database of the present invention stores the user's email, gender, age, country, and health information, as well as the user's choices in session options, allowing data use for research and allowing notifications. The user is able to modify their profile picture, description, first name, last name, gender, country, health information, choice of session options, choice of data use for research and choice of in-app notifications, all of which is stored and updated in the database.

Further, the present invention allows data concerning the acupressure treatments and effects of patent wellness to be shared with a treating physician or health professional to provide real time information for patent care. The present invention also collects data from multiple users and collects real world data to aid further research in the benefits of acupressure—through the PROMIS measures—validated to accurately measure symptoms.

The present invention allows both the health care provider and the patient to monitor the response to acupressure, compare results from treatment variations, and when linked with devices such as smart watches, it can track patient activity and sleep. The present invention will appropriately guide patients to self-administer acupressure, and monitor their acupressure “fidelity” or “compliance”—are they doing it and “how much” and how this affects outcomes (i.e. fatigue, sleep, pain, anxiety, depression) to enhance knowledge and treatment modalities.

What is claimed is:

1. A process for assisting patients with self-application of acupressure, including:
collecting personal data from the patient relating to adverse symptoms;
instructing the patient on procedures for self-administration of acupressure;
recording the patient's symptom response to acupressure treatment to provide treatment data;
forwarding treatment data for data collection; and
organizing treatment data to present feedback to the patient of results of several treatments of self-application of acupressure.

2. A process for assisting patients with self-application of acupressure as set forth in claim 1, including collecting treatment data from multiple patents.

3. A process for assisting patients with self-application of acupressure as set forth in claim 2, wherein said symptoms are sleep and activity levels.

4. A process for assisting patients with self-application of acupressure as set forth in claim 3, including presenting to the patient a graphic representation of the feedback of treatment data over a period of time.

5. A process for assisting patients with self-application of acupressure as set forth in claim 1, wherein said symptoms are sleep and activity levels.

6. A process for assisting patients with self-application of acupressure as set forth in claim 5, including presenting to the patient a graphic representation of the feedback of treatment data over a period of time.

7. A process for assisting patients with self-application of acupressure as set forth in claim 1, including presenting to the patient a graphic representation of the feedback of treatment data personal data over a period of time.

8. A process for assisting patients with self-application of acupressure as set forth in claim 1, including instructing the patient on procedures for self-administration of acupressure in both stimulating acupressure or relaxing acupressure.

9. A process for assisting patients with self-application of acupressure as set forth in claim 8, including instructing the patient on procedures for self-administration of aromatherapy during acupressure.

10. A process for assisting patients with self-application of acupressure as set forth in claim 1, including instructing the patient on procedures for self-administration of aromatherapy during acupressure.

11. A software application for assisting patients with self-application of acupressure, said software application configured to include:
collecting personal data from the patient relating to adverse symptoms;
instructing the patient on procedures for self-administration of acupressure;
recording the patient's symptom response to acupressure treatment to provide treatment data;
forwarding treatment data for data collection; and
organizing treatment data to present feedback to the patient of results of several treatments of self-application of acupressure.

12. A software application for assisting patients with self-application of acupressure as set forth in claim 11, including collecting treatment data from multiple patents.

13. A software application for assisting patients with self-application of acupressure as set forth in claim 12, wherein said symptoms are sleep and activity levels.

14. A software application for assisting patients with self-application of acupressure as set forth in claim 13, including presenting to the patient a graphic representation of the feedback of treatment data over a period of time.

15. A software application for assisting patients with self-application of acupressure as set forth in claim 11, wherein said symptoms are sleep and activity levels.

16. A software application for assisting patients with self-application of acupressure as set forth in claim 15, including presenting to the patient a graphic representation of the feedback of treatment data over a period of time.

17. A software application for assisting patients with self-application of acupressure as set forth in claim 11, including presenting to the patient a graphic representation of the feedback of treatment data personal data over a period of time.

18. A software application for assisting patients with self-application of acupressure as set forth in claim 11, including instructing the patient on procedures for self-administration of acupressure in both stimulating acupressure or relaxing acupressure.

19. A software application for assisting patients with self-application of acupressure as set forth in claim 18, including instructing the patient on procedures for self-administration of aromatherapy during acupressure.

20. A software application for assisting patients with self-application of acupressure as set forth in claim 11, including instructing the patient on procedures for self-administration of aromatherapy during acupressure.
